(19) United States
(12) Patent Application Publication
(10) Pub. No.: US 2024/0369526 A1
(43) Pub. Date: Nov. 7, 2024

(54) INTEGRATED PRINTED SENSOR PATCH FOR MICRO-CLIMATE MONITORING OF GREENHOUSE AND SOIL-RELATED VARIABLES

(71) Applicant: Qatar Foundation for Education, Science and Community Development, Doha (QA)

(72) Inventors: Amine Bermak, Doha (QA); Tareq Al Ansari, Doha (QA); Saleem Khan, Doha (QA)

(21) Appl. No.: 18/653,438

(22) Filed: May 2, 2024

Related U.S. Application Data
(60) Provisional application No. 63/463,448, filed on May 2, 2023.

(51) Int. Cl. G01N 33/24; G01K 7/16; G01N 33/00

(52) U.S. Cl. CPC G01N 33/245; G01K 7/16; G01N 33/004; G01N 33/0047; G01N 33/246

(57) ABSTRACT

A printed sensor patch for micro-climate monitoring is provided. The printed sensor patch includes a substrate having a first portion and second portion, a data acquisition system, and a sensor array. The sensor array includes a moisture level sensor, a humidity sensor, a temperature sensor, and a volatile organic compound sensor.

### PRIORITY CLAIM AND CROSS-REFERENCE TO RELATED APPLICATIONS

The present application claims the benefit of U.S. Provisional Application No. 63/463,448 filed May 2, 2023, which is incorporated herein by reference in its entirety.

### BACKGROUND

With the development of smart agriculture systems, the demand for systems and devices that collect plant status information during their growth, transportation, and storage periods has increased. In an example, sensor data from both the ambient environment and the soil within a greenhouse system can be collected for feedback control of the growth system. This real-time monitoring of the micro-climate inside a greenhouse, as well as of the soil-related variables, may improve the accuracy of irrigation management. In turn, a smart irrigation management system may lead to an increase in crop production with a decrease in labor inputs.

Thus, systems and methods are needed to monitor and collect plant information for smart agriculture systems.

### SUMMARY

In light of the disclosure herein and without limiting the disclosure in any way, in a first aspect of the present disclosure, which may be combined with any other aspect listed herein unless specified otherwise, a printed sensor patch for micro-climate monitoring is provided. The printed sensor patch includes a substrate having a first portion and a second portion, a data acquisition system, and a sensor array having a moisture level sensor, a humidity sensor, a temperature sensor, and a volatile organic compound sensor.

In accordance with a second aspect of the present disclosure, which may be used in combination with any other aspect listed herein unless stated otherwise, the moisture level sensor is disposed on the first portion of the substrate, and the first portion of the substrate is configured to be inserted into soil.

In accordance with a third aspect of the present disclosure, which may be used in combination with any other aspect listed herein unless stated otherwise, the moisture level sensor is configured to monitor a soil water level when the first portion of the substrate is inserted into soil.

In accordance with a fourth aspect of the present disclosure, which may be used in combination with any other aspect listed herein unless stated otherwise, the humidity sensor is disposed on the second portion of the substrate, and the second portion of the substrate extends above a soil surface when the first portion of the substrate is inserted into the soil.

In accordance with a fifth aspect of the present disclosure, which may be used in combination with any other aspect listed herein unless stated otherwise, the humidity sensor is configured to monitor a humidity level.

In accordance with a sixth aspect of the present disclosure, which may be used in combination with any other aspect listed herein unless stated otherwise, the temperature sensor is disposed on the second portion of the substrate, and the second portion of the substrate extends above a soil surface when the first portion of the substrate is inserted into soil.

In accordance with a seventh aspect of the present disclosure, which may be used in combination with any other aspect listed herein unless stated otherwise, the temperature sensor is a resistive temperature detector configured to monitor a temperature.

In accordance with an eight aspect of the present disclosure, which may be used in combination with any other aspect listed herein unless stated otherwise, the volatile organic compound sensor is disposed on the second portion of the substrate, and the second portion of the substrate extends above a soil surface when the first portion of the substrate is inserted into soil.

In accordance with a ninth aspect of the present disclosure, which may be used in combination with any other aspect listed herein unless stated otherwise, the volatile organic compound sensor is configured to identify a volatile organic compound.

In accordance with a tenth aspect of the present disclosure, which may be used in combination with any other aspect listed herein unless stated otherwise, the sensor array further includes a CO₂ sensor and an oxygen sensor.

In accordance with an eleventh aspect of the present disclosure, which may be used in combination with any other aspect listed herein unless stated otherwise, a method of manufacturing a sensor patch for micro-climate monitoring is provided. The method includes printing a sensor array on a polymeric substrate, wherein the sensor array includes a moisture level sensor, a humidity sensor, a temperature sensor, and a volatile organic compound sensor; and connecting the sensor array to a data acquisition system.

In accordance with a twelfth aspect of the present disclosure, which may be used in combination with any other aspect listed herein unless stated otherwise, printing the temperature sensor on the polymeric substrate includes printing a plurality of nanomaterial-based metal interdigital electrodes.

In accordance with a thirteenth aspect of the present disclosure, which may be used in combination with any other aspect listed herein unless stated otherwise, each of the plurality of nanomaterial-based metal interdigital electrodes are separated by an equal distance.

In accordance with a fourteenth aspect of the present disclosure, which may be used in combination with any other aspect listed herein unless stated otherwise, a nanoparticle-based metallic ink is used to print the humidity sensor.

In accordance with a fifteenth aspect of the present disclosure, which may be used in combination with any other aspect listed herein unless stated otherwise, the humidity sensor includes a humidity sensing layer and a plurality of interdigital electrodes, wherein the humidity sensing layer is disposed above the plurality of interdigital electrodes.

In accordance with a sixteenth aspect of the present disclosure, which may be used in combination with any other aspect listed herein unless stated otherwise, printing the volatile organic compound sensor includes printing a plurality of interdigital electrodes, wherein each one of the plurality of interdigital electrodes have a pitch size.

In accordance with a seventeenth aspect of the present disclosure, which may be used in combination with any other aspect listed herein unless stated otherwise, the sensing layer is disposed above the plurality of interdigital electrodes.

In accordance with an eighteenth aspect of the present disclosure, which may be used in combination with any other aspect listed herein unless stated otherwise, the method further includes testing the sensing layer with a volatile organic compound.

In accordance with a nineteenth aspect of the present disclosure, which may be used in combination with any other aspect listed herein unless stated otherwise, the moisture level sensor includes a parallel plate structure having at least two electrodes.

In accordance with a twentieth aspect of the present disclosure, which may be used in combination with any other aspect listed herein unless stated otherwise, the moisture level sensor further includes a protective film.

Additional features and advantages of the disclosed method and apparatus are described in, and will be apparent from, the following Detailed Description and the Figures. The features and advantages described herein are not all-inclusive and, in particular, many additional features and advantages will be apparent to one of ordinary skill in the art in view of the figures and description. Moreover, it should be noted that the language used in the specification has been principally selected for readability and instructional purposes, and not to limit the scope of the inventive subject matter.

### BRIEF DESCRIPTION OF THE FIGURES

Understanding that figures depict only typical embodiments of the invention and are not to be considered to limit the scope of the present disclosure, the present disclosure is described and explained with additional specificity and detail through the use of the accompanying figures. The figures are listed below.

FIG. 1 illustrates a schematic diagram of an integrated sensor patch, according to an example embodiment of the present disclosure.

FIG. 2 illustrates a schematic diagram of a sensor array and a communication system, according to an example embodiment of the present disclosure.

FIG. 3, views (a) and (b), illustrates a schematic diagram of a moisture level sensor, according to an example embodiment of the present disclosure.

FIG. 4, views (a) and (b), illustrates a schematic diagram of a humidity sensor, according to an example embodiment of the present disclosure.

FIG. 5, views (a) and (b), illustrates a schematic diagram of a temperature sensor, according to an example embodiment of the present disclosure.

FIG. 6 illustrates a sensor array and a data acquisition and communication module, according to an example embodiment of the present disclosure.

FIG. 7 illustrates experimental results of soil moisture level when comparing time to resistance, according to an example embodiment of the present disclosure.

FIG. 8 illustrates experimental results of sensor resistance in response to temperature variation, according to an example embodiment of the present disclosure.

FIG. 9 illustrates experimental results of sensor resistance in response to humidity variation cycles, according to an example embodiment of the present disclosure.

FIG. 10 illustrates experimental results of sensor resistance in response to a presence of different volatile organic compounds, according to an example embodiment of the present disclosure.

### DETAILED DESCRIPTION

The following system generally relates to an integrated printed sensor patch for real-time monitoring of micro-climate conditions inside a greenhouse as well as soil-related variables. As discussed above, sensor data from both the ambient environment and the soil within, for example, a greenhouse system can be collected for feedback control of a growth system. Sensor data may be relevant to several variables, including soil moisture, the temperature of the ambient environment, the humidity of the ambient environment, or the amount of volatile organic compounds (“VOCs”). Collecting the sensor data for feedback control of a growth system may lead to an increase of crop production with minimal labor costs. Further, the integrated printed sensor patch of the present disclosure provides a low-cost system for deployment over large areas.

In an example embodiment, the printed sensor patch is developed on a single substrate, which can be attached to a 3D printed rigid support through lamination. The sensor patch developed on a single substrate may be configured to monitor variables related to the soil or the ambient environment surrounding the soil. For example, the printed sensor patch may be configured to monitor moisture levels of the soil and the humidity level, the temperature, and the VOCs of the ambident environment surrounding the soil. In one embodiment, the ambient environment may correspond to the inside of a greenhouse system.

The sensor patch may include a rigid support, a sensor array, and a data acquisition system. The sensor array further includes a moisture level sensor, a humidity sensor, a temperature sensor, and a VOCs sensor. These sensors are developed as a single patch, which adheres to a perforated 3D printed support, part of which is inserted inside the soil including the moisture sensor, whereas the remaining three sensors are left exposed to the outer environment. In use, real-time data is acquired, processed, and stored in an onsite system, which may be wirelessly transmitted to a remote handheld device through, for example, a Bluetooth connection. The data acquisition system includes a lightweight data acquisition module and a signal conditioning circuit module. The data acquisition system is configured to record and analyze the real-time data, which can be stored in the monitoring system. In an embodiment, the data acquisition system processes real-time sensor data with minimal interruption form the ambient environment.

In an example application, printed sensor patches are manufactured and deployed to create a smart irrigation management system. Namely, the sensor patches may be deployed at distributed locations in a target agricultural field at specific depths beneath the surface of the soil. The sensor patch may then monitor the available water content of the soil at each location through a change in dielectric permittivity. A threshold level can be set for ideal conditions and below that, the irrigation system will be activated to provide water at desired levels. The temperature sensor may be used as a supporting signal, authenticating the need of irrigation. The level of the soil moisture and temperature are related, and, at higher temperatures, there is higher probability of less water content due to the evaporative mechanism from soil surface. This ultimately leads to reducing the level of the soil moisture and would be used as an authentication signal for activating the irrigation system. Therefore, a combination of these two sensors would allow easy, rapid, and effective monitoring of the soil as well as crop health in real-time. The humidity and VOCs sensors could help in monitoring and detecting the start of the food ripening inside a greenhouse and spoilage, especially in the storage rooms.

To save costs, the printed sensor patch may be produced with printing technology for creating a smart irrigation system. For example, a low-cost, material-efficient inkjet with screening technologies would produce the required components on a polymeric substrate having, for example, a plurality of resistance-based sensors. In an illustrative example, variations in the electrical resistance-based sensors would be deployed at distributed potential spots in the targeted agricultural fields at specific depths beneath the ground surface. The change in dielectric permittivity linked to the bulk electrical resistance of the parallel plate sensing structure would be correlated with the available water content of the soil.

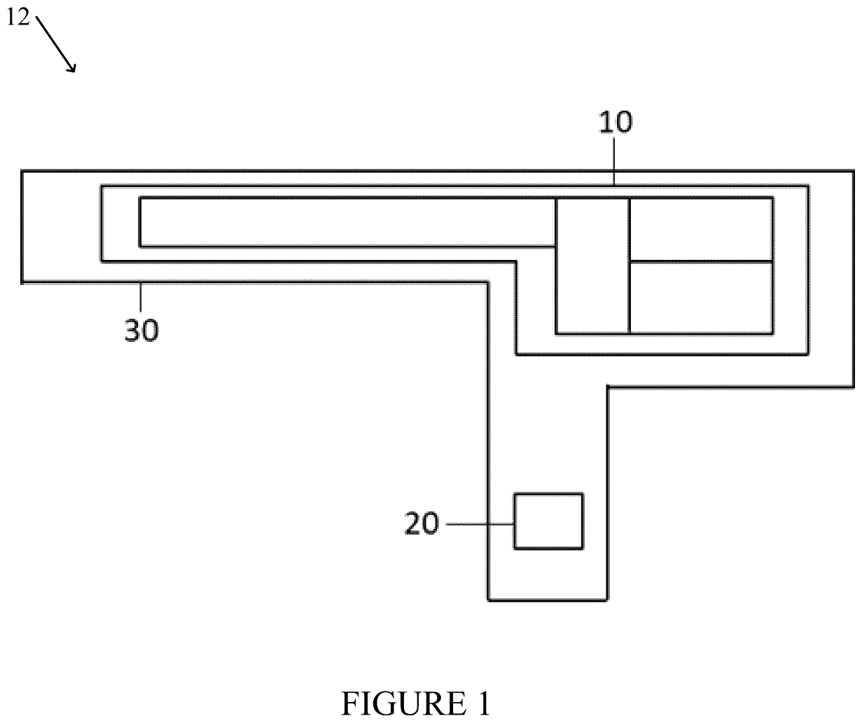

FIG. 1 illustrates a schematic diagram of an integrated sensor patch, according to an example embodiment of the present disclosure. The system 12 includes a sensor array 10 and a data acquisition system 20. The sensor array 10 and the data acquisition system 20 are disposed on a rigid support 30, which may be biocompatible. The sensor array 10 may include a plurality of sensors interconnected to signal conditioning circuits to eliminate redundant data generated through the exposure to the surrounding environment. In many embodiments, the rigid support 30 is 3D printed. With the ability to 3D print the rigid support 30, the shape of the rigid support 30 can be easily altered to complement the design of the sensor array 10 or the data acquisition system 20. For example, the number, shape, or design of the sensor array 10 can be customized and then the rigid support 30 can be printed to support the sensor array 10.

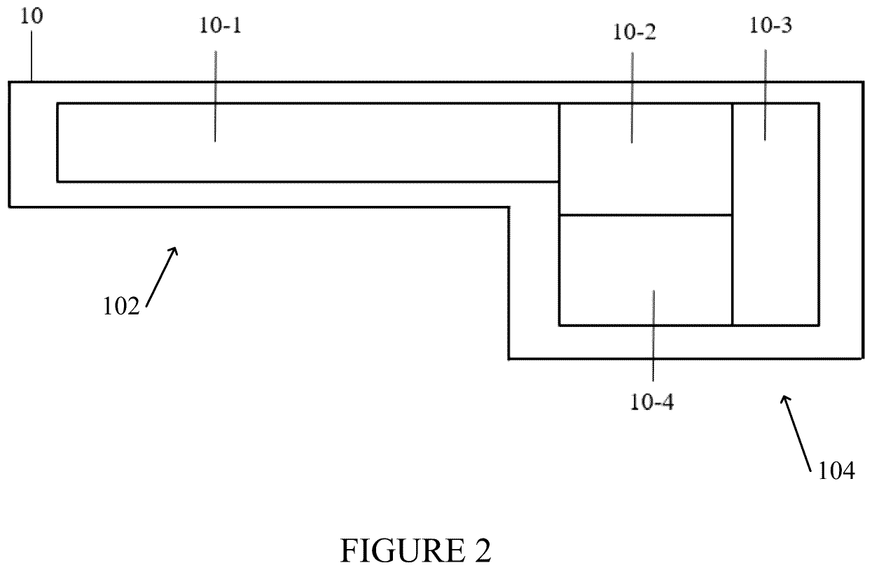

FIG. 2 illustrates a schematic diagram of a sensor array and a communication system, according to an example embodiment of the present disclosure. The sensor array 10 includes a moisture level sensor 10-1, a humidity sensor 10-2, a temperature sensor 10-3, and a VOCs sensor 10-4. In various embodiments, each sensor monitors variables of the ambient environment or the soil. For example, the moisture level sensor 10-1 may measure the level of moisture within the soil. As such, the moisture level sensor 10-1 is located on a first portion 102 of the sensor array 10 that is inserted into the soil. In reference to FIG. 2, the moisture level sensor 10-2 is disposed on the first portion 102, which is inserted into the soil.

The humidity sensor 10-2, the temperature sensor 10-3, and the VOCs sensor 10-4 measure respective variables of the ambient environment around the soil. In the embodiment of FIG. 2, the humidity sensor 10-2, the temperature sensor 10-3, and the VOCs sensor 10-4 are disposed on a second portion 104 of the sensor array 10. When the first portion 102 of the sensor array 10 is inserted into the soil, the second portion 104 of the sensor array 10 extends from the surface of the soil to measure the ambient environment. It should be appreciated that any number and types of sensors can be included on the sensor array to monitor variables. For example, the sensor array 10 may further include a CO₂ sensor and an oxygen sensor.

The VOCs sensor 10-4 may be printed for measuring the levels of volatile organic compounds present inside a micro-climatic closed environment. In an example embodiment, the VOCs sensor 10-4 may be advantageous to indicate the existence of VOCs inside a greenhouse environment or a food storage facility. VOCs may be generated from over-ripening or spoilage of food items inside the greenhouse environment or the food storage facility. Continuous monitoring for such conditions is desired for a timely action to avoid significant losses. The VOCs sensor 10-4 may be developed by printing interdigital electrodes at specific pitch sizes, which are covered by a VOC sensing layer coated by using a screen-printing technology. The sensing layer may be tested and calibrated at lab conditions, where different levels of acetone, ethanol, and isopropanol as representative VOCs are tested in a closed test chamber. Different volumes of these VOCs are injected inside the chamber to generate a certain amount of climatic condition in part per million (“ppm”) ranges. The lab level tests may result in a fast response where the rise time of approximately one and a half minutes is recorded for above 60% of the cumulative resistance change for all the three types of VOCs.

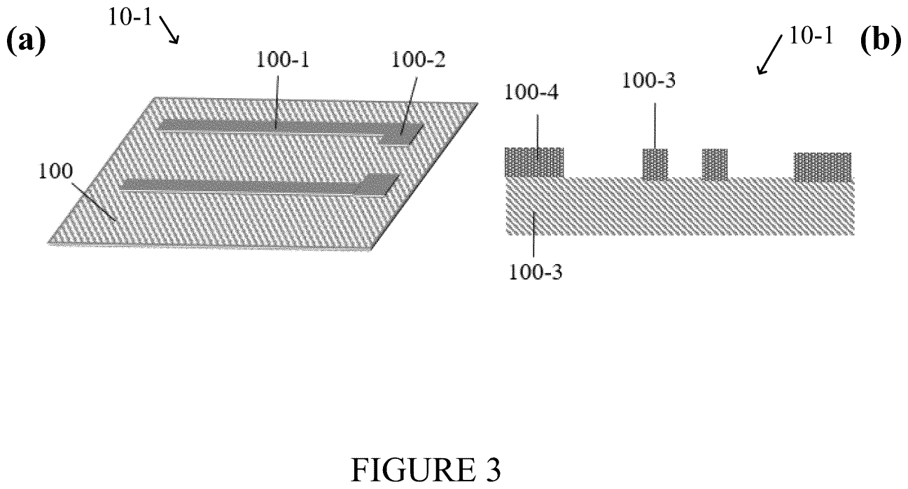

FIG. 3, views (a) and (b), illustrates a schematic diagram of a moisture level sensor, according to an example embodiment of the present disclosure. FIG. 3, view (a), illustrates a perspective view of the moisture level sensor, and FIG. 3, view (b), illustrates a cross-sectional view of the moisture level sensor. The moisture level sensor 10-1 includes a substrate 100, a plurality of parallel plate interdigital electrodes 100-1, and a plurality of contacting pads 100-2. In reference to the cross-sectional view of view (b), the moisture level sensor 10-1 includes the substrate 100, the plurality of parallel plate interdigital electrodes 100-3, and the plurality of contacting pads 100-4. The moisture level sensor 10-1 may provide advantageous monitoring within, for example, a greenhouse that covers a large area. By monitoring the moisture of the soil through the monitoring system, resources and human intervention could be minimized. For example, the system connected to the main control system may automatically start watering cycles in response to the data obtained from the moisture level sensor 10-1.

In an example embodiment, the moisture level sensor 10-1 may be printed for measuring the water or moisture level of the soil. The moisture level sensor 10-1 is made in a parallel plate structure, where the two long electrodes (˜10 mm long) are used to cover the soil near the roots. A nanoparticle-based metallic ink paste is used to develop the sensor geometry, including the geometry for the moisture level sensor 10-1. The variation in the bulk electrical resistance is triggered by the change in the water level inside the soil. In an embodiment, the moisture level sensor 10-1 may be further covered by a thin, porous film. Such protective film may enhance the mechanical stability and the robustness of the moisture level sensor 10-1 when placed inside the soil. This may increase the reliability of the recorded data as well as long term stable operation of the moisture level sensor 10-1 inside the soil at different moisture conditions.

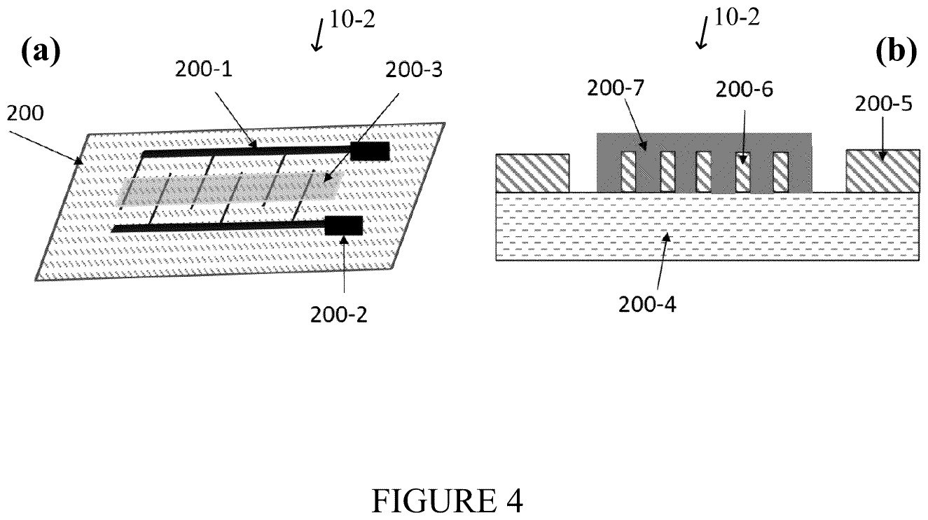

FIG. 4, views (a) and (b), illustrates a schematic diagram of a humidity sensor, according to an example embodiment of the present disclosure. The humidity sensor 10-2 may indicate the hydration level inside the micro-climatic environment inside a greenhouse. The humidity sensor may play a significant role by monitoring the levels in, for example, a food storage facility showing a critical level of hydration level that may lead to the spoilage of food items. FIG. 4, view (a), illustrates a perspective view of the humidity sensor 10-2, and FIG. 4, view (b), illustrates a cross-sectional view of the humidity sensor 10-2. The humidity sensor 10-2 includes a substrate 200, a plurality of parallel plate interdigital electrodes 200-1, a plurality of contacting pads 200-2, and a sensing film 200-3. In reference to the cross-sectional view of FIG. 4, the humidity sensor 10-2 includes a substrate 200-4, the plurality of parallel plate interdigital electrodes 200-6, the plurality of contacting pads 200-5, and the sensing film 200-7.

In an example embodiment, the humidity sensor 10-2 is printed on a plastic substrate, and the humidity sensor 10-2 is configured to provide data regarding the hydration level inside, for example, the greenhouse micro-climate environment. The humidity sensor 10-2 structure includes a plurality of parallel plate interdigital electrodes 200-1 covered with a sensing film 200-3. Further, nanoparticle-based metallic ink is patterned using inkjet printing technology, whereas the spacing between the electrodes 200-1 is covered with a nanocomposite material sensitive to slight variations in the humidity. The nanocomposite based thin film is highly sensitive with a quick response and recovery time of about 0.25 and 0.35 seconds, respectively, under standard temperature and pressure. This high-speed recovery time may be desired for monitoring the humidity level inside a micro-climatic environment, which could change abruptly under each watering cycle of the plants. This printed sensor may be highly sensitive of 96.36% in detectable range from 5% to 95% RH, which has negligible cross sensitivity from other constituents in air due to the distinguished properties of the nanocomposite layer. The interaction of OH— molecules with the nanocomposite is highly detectable as the electrical resistance of the sensor goes down by completing the current paths with OH— molecules.

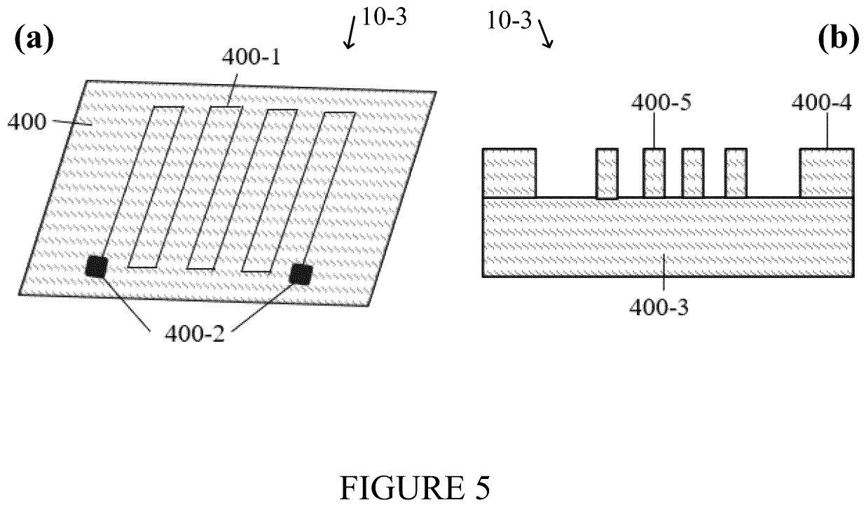

FIG. 5, views (a) and (b), illustrates a schematic diagram of a temperature sensor, according to an example embodiment of the present disclosure. FIG. 5, view (a), illustrates a perspective view of the temperature sensor 10-3, and FIG. 5, view (b), illustrates a cross-sectional view of the temperature sensor 10-3. The temperature sensor 10-3 includes a substrate 400, an electrically conductive pattern 400-1, and a plurality of contacting pads 400-2. The cross-sectional view of the temperature sensor is also shown, depicting the substrate 400-3, the plurality of contacting pads 400-4, and the electrically conductive pattern 400-5.

In an example embodiment, the temperature sensor 10-3 is developed by printing nanomaterials-based metal interdigital electrodes (“IDEs”), which are filled with the temperature sensing layer. Equal spacing between the electrodes is maintained to ensure containment of the sensing layer and exposure to the detection event without being interrupted by the surrounding environment. The interconnection and pads are also printed by using the same metallic nanoparticles-based ink for the readout. A thin encapsulate layer is applied on the sensing layer as well as on the metal electrodes. The encapsulate layer may stabilize device performance as the metal electrodes are prone to oxidation and the temperature sensing layer is sensitive to humidity. The encapsulate layer is applied on the whole sensing area using a screen-printing technology. Change in the bulk electrical resistance is caused by the temperature variations, which is first calibrated in an oven at different thermal cycles.

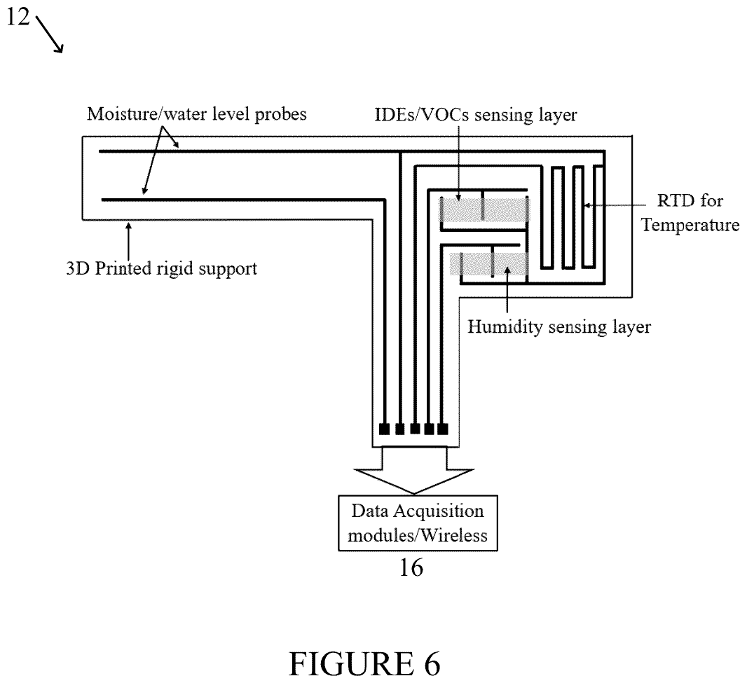

FIG. 6 illustrates a sensor array and a data acquisition and communication module, according to various examples of the present disclosure. The communication module includes a power management and charging unit. The communication module further includes a microcontroller for the signal conditioning and processing. All sensors are connected to the data acquisition board 16 and data is taken and processed in real-time followed by the data transmission unit. The data acquisition and communication module may be configured to record and analyze the real-time data, which can be stored in the system. The small size, light weight, and portable nature of the whole acquisition and communication module may process the data with minimal interruption form the ambient environment. The data acquisition module may be connected to a desktop computer (not shown) or may further include a low power wireless transmission module connected to a microcontroller to transmit the sensor data to a personalized portable system.

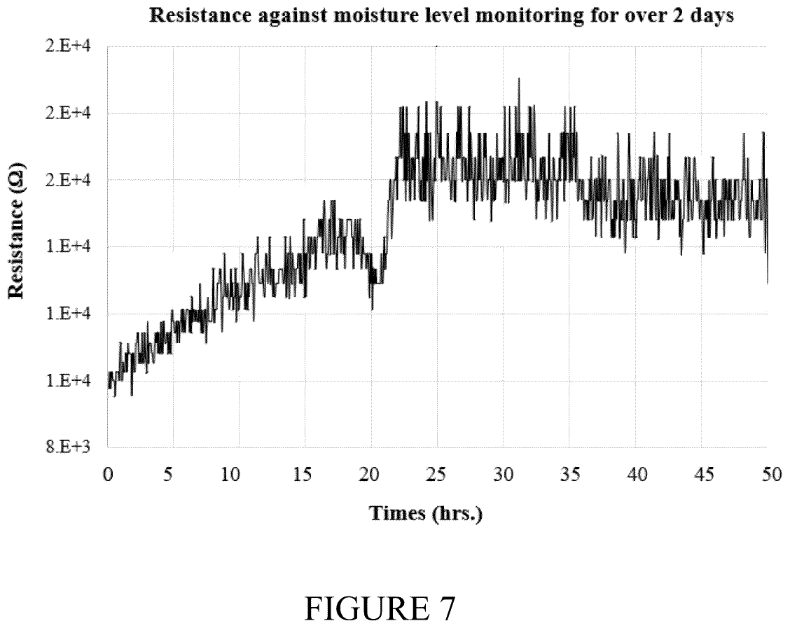

FIG. 7 illustrates experimental results of soil moisture level when comparing time to resistance, according to various examples of the present disclosure. In reference to FIG. 7, resistance (Ω) on the y-axis is plotted against time (hours) on the x-axis. These variables were recorded for 50 hours to compare resistance to moisture. The data illustrated in FIG. 7 may be recorded by connecting the moisture sensor 10-1 to a source meter and recording real-time data. Moreover, the data may be collected for a longer period, starting when the soil is filled with water to the level covering the plant roots and ending when a certain level of water is consumed or evaporated. This causes the change in the medium contacting the two parallel plate conductive tracks and thus results in a change in electrical response.

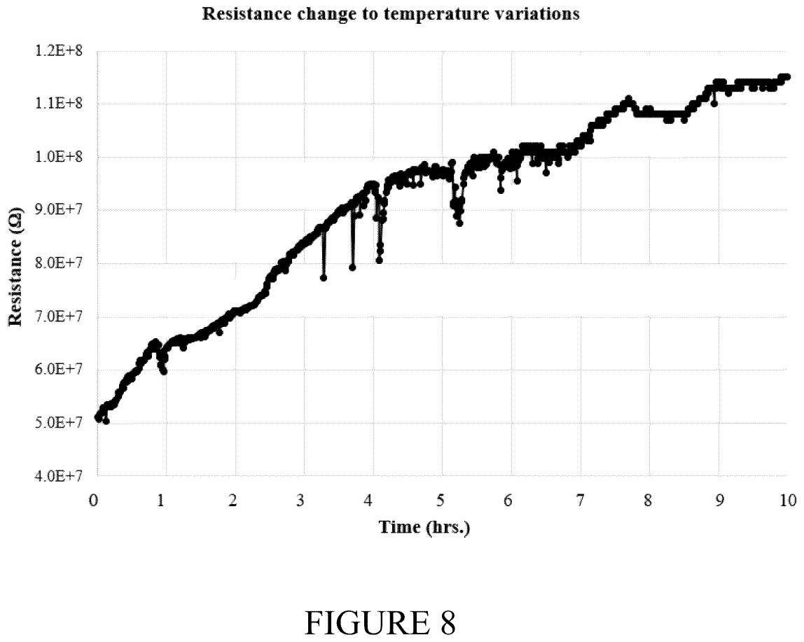

FIG. 8 illustrates experimental results of sensor resistance in response to temperature variation, according to various examples of the present disclosure. In reference to FIG. 8, resistance (Ω) on the y-axis is plotted against time (hours) on the x-axis. These variables were recorded for 50 hours to compare resistance to temperature. For testing and calibrating, the sensors were placed inside the oven and the corresponding change in electrical resistance in response to temperature increase was recorded. Like the approach with the moisture sensor 10-1, the temperature sensor 10-3 connects to a source meter for recording the data in real-time.

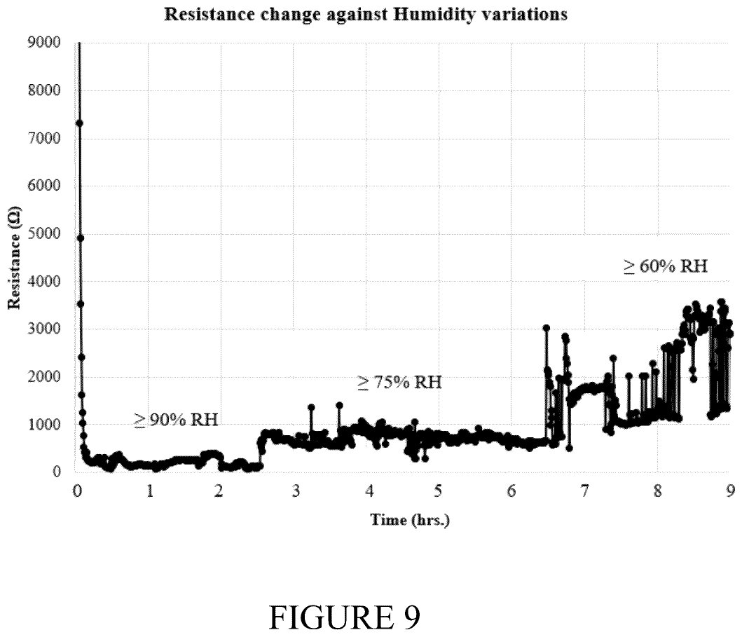

FIG. 9 illustrates experimental results of sensor resistance in response to humidity variation cycles, according to various examples of the present disclosure. In reference to FIG. 9, resistance (Ω) on the y-axis is plotted against time (hours) on the x-axis. These variables were recorded for 9 hours to determine the resistance change against humidity variations. The resulting data provides example humidity variations inside the microclimatic environment. In particular, the sensors are exposed to different levels of humidity inside an oven to mimic micro-climatic conditions and data is recorded in real-time by recording the signal in resistance variations against the humidity variations. Sensors were tested at three different humidity conditions, i.e. ≥(90%, 75% and 60%) relative humidity (“RE”).

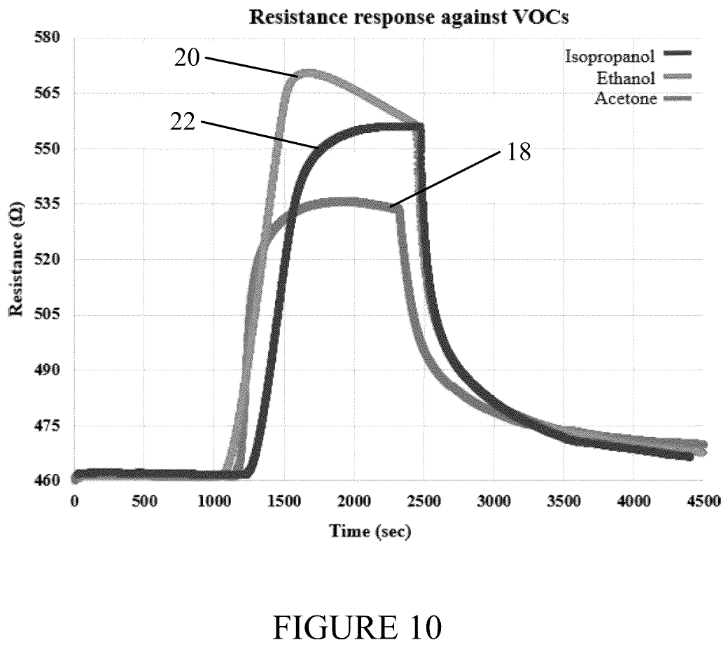

FIG. 10 illustrates experimental results of sensor resistance in response to a presence of different volatile organic compounds, according to various examples of the present disclosure. In reference to FIG. 10, resistance (Q) on the y-axis is plotted against time (seconds) on the x-axis. The resistance was recorded for 4500 seconds to determine the resistance change against different volatile organic compounds. The resulting data provides details about the experimental results of VOC sensor 10-4 tested in the presence of three different VOCs. The VOCs sensor 10-4 was tested in a closed chamber. Then, a specific concentration of each VOC is introduced inside the chamber and data is recorded in the form of electrical resistance change against the level of concentrations of the VOC. In FIG. 10, the three curves in the graph represent three different VOCs tested, i.e. acetone 18, ethanol 22, and isopropanol 20 as representative VOCs to benchmark for real-time testing.

Although the system has been described in certain specific aspects, many additional modifications and variations would be apparent to those skilled in the art. In particular, any of the various processes described above can be performed in alternative sequences and/or in parallel in order to achieve similar results in a manner that is more appropriate to the requirements of a specific application. It is therefore to be understood that the present disclosure can be practiced otherwise than specifically described without departing from the scope and spirit of the present embodiments. Thus, embodiments of the present invention should be considered in all respects as illustrative and not restrictive. It will be evident to the annotator skilled in the art to freely combine several or all of the embodiments discussed here as deemed suitable for a specific application of the invention. Throughout this disclosure, terms like “advantageous”, “exemplary” or “preferred” indicate elements or dimensions which are particularly suitable (but not essential) to the invention or an embodiment thereof and may be modified wherever deemed suitable by the skilled annotator, except where expressly required. Accordingly, the scope of the invention should be determined not by the embodiments illustrated, but by the appended claims and their equivalents.

The invention is claimed as follows:

1: A printed sensor patch for micro-climate monitoring, comprising:
a substrate having a first portion and a second portion;
a data acquisition system; and
a sensor array having a moisture level sensor, a humidity sensor, a temperature sensor, and a volatile organic compound sensor.

2: The printed sensor patch of claim 1, wherein the moisture level sensor is disposed on the first portion of the substrate, and the first portion of the substrate is configured to be inserted into soil.

3: The printed sensor patch of claim 2, wherein the moisture level sensor is configured to monitor a soil water level when the first portion of the substrate is inserted into the soil.

4: The printed sensor patch of claim 1, wherein the humidity sensor is disposed on the second portion of the substrate, and the second portion of the substrate extends above a soil surface when the first portion of the substrate is inserted into soil.

5: The printed sensor patch of claim 4, wherein the humidity sensor is configured to monitor a humidity level.

6: The printed sensor patch of claim 1, wherein the temperature sensor is disposed on the second portion of the substrate, and the second portion of the substrate extends above a soil surface when the first portion of the substrate is inserted into soil.

7: The printed sensor patch of claim 6, wherein the temperature sensor is a resistive temperature detector configured to monitor a temperature.

8: The printed sensor patch of claim 1, wherein the volatile organic compound sensor is disposed on the second portion of the substrate, and the second portion of the substrate extends above a soil surface when the first portion of the substrate is inserted into soil.

9: The printed sensor patch of claim 8, wherein the volatile organic compound sensor is configured to identify a volatile organic compound.

10: The printed sensor patch of claim 1, wherein the sensor array further comprises a CO₂ sensor and an oxygen sensor.

11: A method of manufacturing a sensor patch for micro-climate monitoring, comprising:
printing a sensor array on a polymeric substrate, wherein the sensor array includes a moisture level sensor, a humidity sensor, a temperature sensor, and a volatile organic compound sensor; and
connecting the sensor array to a data acquisition system.

12: The method of manufacturing the sensor patch of claim 11, wherein printing the temperature sensor on the polymeric substrate includes printing a plurality of nanomaterial-based metal interdigital electrodes.

13: The method of manufacturing the sensor patch of claim 12, wherein each of the plurality of nanomaterial-based metal interdigital electrodes are separated by an equal distance.

14: The method of manufacturing the sensor patch of claim 11, wherein a nanoparticle-based metallic ink is used to print the humidity sensor.

15: The method of manufacturing the sensor patch of claim 14, wherein the humidity sensor comprises a humidity sensing layer and a plurality of interdigital electrodes, wherein the humidity sensing layer is disposed above the plurality of interdigital electrodes.

16: The method of manufacturing the sensor patch of claim 11, wherein printing the volatile organic compound sensor includes printing a plurality of interdigital electrodes, wherein each one of the plurality of interdigital electrodes have a pitch size.

17: The method of manufacturing the sensor patch of claim 16, wherein the volatile organic compound sensor comprises a sensing layer, wherein the sensing layer is disposed above the plurality of interdigital electrodes.

18: The method of manufacturing the sensor patch of claim 17, further comprising testing the sensing layer with a volatile organic compound.

19: The method of manufacturing the sensor patch of claim 11, wherein the moisture level sensor comprises a parallel plate structure having at least two electrodes.

20: The method of manufacturing the sensor patch of claim 19, wherein the moisture level sensor further comprises a protective film.
